(19) United States
(12) Patent Application Publication
(10) Pub. No.: US 2026/0315575 A1
(43) Pub. Date: Oct. 15, 2026

(54) TYROSINASE-BASED TRANSDERMAL DELIVERY SYSTEM FOR TREATING VITILIGO

(71) Applicant: SHANGHAI JIAO TONG UNIVERSITY, Shanghai (CN)

(72) Inventors: Xin Jin, Shanghai (CN); Jixiang Li, Shanghai (CN); Xinyuan Zhu, Shanghai (CN)

(21) Appl. No.: 19/683,667

(22) Filed: May 20, 2026

Related U.S. Application Data
(63) Continuation of application No. PCT/CN2024/095494, filed on May 27, 2024.

(30) Foreign Application Priority Data
Dec. 13, 2023 (CN) 202311704896.7

(51) Int. Cl. A61K 38/44; A61K 9/00; A61K 9/50; A61K 9/51; A61K 31/01; A61K 31/122; A61K 31/352; A61K 31/355; A61K 31/7048; A61P 17/00

(52) U.S. Cl. CPC A61K 38/44; A61K 9/0014; A61K 9/5026; A61K 9/5192; A61K 31/01; A61K 31/122; A61K 31/352; A61K 31/355; A61K 31/7048; A61P 17/00; C12Y 114/18001

(57) ABSTRACT

Provided is a tyrosinase-based transdermal delivery system for treating vitiligo, having a core-shell structure, a core being tyrosinase, and a shell being a polymer shell. Double bonds are introduced into tyrosinase, and the surface of protein is coated with a polymer protection layer through in-situ polymerization to protect the activity of the tyrosinase, and promote the transdermal delivery of the tyrosinase. Tyrosinase nanogel can be controlled to be delivered to melanocytes deep in skin and hair follicles in a targeted manner by regulating and controlling polymerization conditions of the polymer shell to promote in-situ synthesis of melanin. The system can also load active small molecules with an anti-inflammatory function at a center of the tyrosinase, and the small molecules are released after being delivered to an inflammatory area of vitiligo in a targeted manner along with the tyrosinase nanogel, thereby relieving inflammation, and protecting the melanocytes from being damaged.

### CROSS-REFERENCE TO RELATED APPLICATIONS

The present application is a continuation application of PCT application No. PCT/CN2024/095494 filed on May 27, 2024, which claims the benefit of Chinese Patent Application No. 202311704896.7 filed on Dec. 13, 2023. The contents of all of the aforementioned applications are incorporated by reference herein in their entirety.

### TECHNICAL FIELD

The present disclosure belongs to the technical field of drug transdermal delivery systems, and particularly relates to a tyrosinase-based nanotransdermal delivery system for treating vitiligo.

### BACKGROUND

Vitiligo is a common pigmentary skin disease, manifested as the loss of local functional melanocytes. This type of disease affects more than 2% of the global population, and has a serious impact on the physical and mental health of patients. Moreover, its etiology is complicated, and a rapid and effective radical cure method of vitiligo is still under research. Several causes have been confirmed to be related to the onset of vitiligo, including oxidative stress, production of inflammatory mediators, autoimmune responses, etc. Based on different pathogenic mechanisms, clinical and commercial drug treatment regimens have been studied to promote local pigmentation in patients with vitiligo. Reducing the damage of functional melanocytes in affected areas is currently a first-line treatment strategy. In view of the attack of melanocytes in the affected areas in the autoimmune response, local transdermal delivery of corticosteroids and calcineurin inhibitors can reduce melanocyte damage through local immunoregulation. However, treatment with immunosuppressive agents may bring systemic adverse reactions, and patients face a higher risk of recurrence of symptoms after drug withdrawal. The destruction of melanocytes in the affected areas caused by oxidative stress is an important pathogenic mechanism. Local delivery of small molecule antioxidants or catalase and superoxide dismutase is also a therapeutic strategy, but this method has shown low patient universality in clinical trials. Anti-inflammatory drugs are usually used in combination with other therapeutic techniques such as phototherapy to show the ability to promote pigmentation, which imposes high requirements on equipment and instruments. In addition to reducing melanocyte damage, directly promoting melanin synthesis in melanocytes is another therapeutic idea to improve pigmentation in the affected areas; and delivery of a tyrosinase activator is a mainstream method. A catalytic step of tyrosinase is a key step in melanin synthesis, and the tyrosinase activator promotes melanin production in situ by increasing the activity of tyrosinase in melanocytes in the affected areas. However, there are certain requirements for the concentration of the activator, and an excessively high concentration of the activator will produce side effects.

Compared with oral administration and intravenous injection, local transdermal administration has the advantages of convenience and non-invasiveness, maintenance of drug activity, targeted delivery, etc., and is especially suitable for the management of diseases such as vitiligo that require local, long-term, and multiple administrations. Therefore, transdermal delivery is generally adopted for the drug treatment of vitiligo. However, the multi-layered and complex structure of skin creates high-efficiency barrier effects including a physical barrier, a chemical barrier, and an immune barrier. Both small molecule drugs or macromolecule drugs face the problems of low penetration efficiency and failure to reach melanocytes in a basal layer of the skin and the bottom of hair follicles to exert their effects during transdermal administration. Due to the high-efficiency barrier effects of the skin, only increasing the drug concentration offers little help in enhancing penetration, but will cause skin irritation or allergy. In addition, most active drugs are hydrophobic substances with low solubility in traditional transdermal preparations, which severely limits the therapeutic effect of the drugs.

In the field of vitiligo treatment, delivery technologies used in locally administered preparations that have been put into clinical use mainly include penetration enhancers (such as ethanol), liposomal formulations, and microneedles. However, the penetration enhancers promote drug penetration by temporarily destroying the skin barrier, which has a greater irritation effect on the skin, and for inflammatory vitiligo, there is a risk of aggravating the condition in the affected area. Liposome-encapsulated drugs typically have a size of more than 200 nm, and the resulting preparations are in a metastable state, making it difficult to meet the requirements of long-term use and resulting in unsatisfactory delivery effects. In addition, these two technologies are mainly used in the delivery of small molecule drugs such as immunosuppressants, which not only have limited help to the transdermal efficiency of active macromolecule drugs such as enzymes, but also the presence of partial penetration enhancers may cause the inactivation of enzymes, thus losing the ability to treat vitiligo. The microneedle technology uses a needle tip with a certain mechanical strength to puncture the skin to form pores, thus weakening the barrier during transdermal drug delivery. However, the treatment of vitiligo requires a long cycle and multiple administrations, and frequent use of microneedles will cause local skin damage and even trigger inflammation. In addition, the depigmentation symptoms of vitiligo originate from the damage and loss of melanocytes, so a transdermal delivery target of vitiligo is melanocytes located in the basal layer of the skin and deep in the hair follicles. However, the existing transdermal delivery technology cannot control the transdermal depth, resulting in low treatment efficiency.

In summary, the treatment of vitiligo in the prior art has the following defects: small molecule drugs target different mechanisms, have low universality and have side effects; and a macromolecular transdermal technology has low efficiency and has side effects.

### SUMMARY

In order to overcome the defects of low universality, large side effects, low local administration efficiency and inability of targeted delivery of a treatment solution for vitiligo in the prior art, the present disclosure proposes a tyrosinase-based transdermal delivery system for promoting in-situ pigmentation, wherein the system takes modified tyrosinase gel as a carrier and an active component, tyrosinase nanogel with a polymer shell is formed through in-situ polymerization, the polymer shell can play a protective role on the activity of tyrosinase, and the surface properties and size of the polymer shell help the nanogel to efficiently penetrate the skin barrier and be delivered to hair follicles and deep skin layers to promote melanin production. In addition, tyrosinase itself can be used as a carrier of anti-inflammatory small molecules. The nanogel can transdermally transport tyrosinase and its loaded small molecule active ingredients to hair follicles and deep skin layers as a whole, playing a synergistic role in the treatment of vitiligo from two aspects of treating local inflammation and promoting in-situ melanin synthesis.

In order to solve the above technical problems, the present disclosure provides the following technical solutions:

provided is a tyrosinase-based transdermal delivery system for treating vitiligo, wherein the system is of a core-shell structure, a core is tyrosinase, and a shell is a polymer shell.

Further, a polymer in the polymer shell may be a polymeric material wrapped around an outer layer of the tyrosinase by a polymerization reaction, and is selected from an acrylamide copolymer, a vinylpyrrolidone copolymer, polyol, polylactic acid, a phosphocholine polymer, a betaine polymer, and chitosan. The polymer can be obtained by polymerization of monomers with carbon-carbon unsaturated double bonds, such as polyacrylamide and polyvinylpyrrolidone, or can be obtained by polycondensation or ring-opening polymerization of monomers with different functional groups; and the polymer can also be a natural polymer coating the surface of tyrosinase by electrostatic attraction, hydrogen bonding, intermolecular interaction, cross-linking, etc. Or the polymer may be a polymeric material wrapped around an outer layer of the tyrosinase by electrostatic interaction and may be a positively or negatively charged polyelectrolyte, wherein the positively charged polyelectrolyte is at least one selected from poly(acrylamine hydrochloride), poly (L-lysine), polyethyleneimine, poly (L-histidine), poly (N,N-dimethylaminoethyl methacrylate), polymethacrylamidopropyltrimethylammonium chloride, and natural or synthetic polysaccharides; and the negatively charged polyelectrolyte is at least one selected from polyacrylic acid, polystyrene sulfonate, alginate, hyaluronic acid, heparin, heparan sulfate, chondroitin sulfate, dextran sulfate, polymethacrylic acid, oxidized cellulose, carboxymethyl cellulose, polyaspartic acid, and polyglutamic acid.

Correspondingly, a monomer of the polymeric material wrapped around the outer layer of the tyrosinase by the polymerization reaction is at least one selected from vinylpyrrolidone, acrylamide, N-propenylsuccinimide, 2-methacryloyloxyethylphosphocholine, [2-(methacryloyloxy)ethyl]dimethyl-(3-sulfopropyl) ammonium hydroxide, polyethylene glycol methyl ether acrylate, ethylene glycol, propylene glycol, butylene glycol, and lactide.

Further, in the core-shell structure, the tyrosinase loaded with small molecules with anti-inflammatory activity as the core has a size of 2-15 nm, preferably 5-10 nm; and the polymer shell as the shell has a thickness of 1-100 nm, preferably 5-50 nm, more preferably 7-20 nm. Further, the tyrosinase-based transdermal delivery system has a surface Zeta potential of −7 mV to +7 mV, preferably a positive potential, such as +4 mV to +6 mV.

When the surface Zeta potential of the tyrosinase-based transdermal delivery system is +4 mV to +6 mV, its penetration depth and the size of nanogel satisfy D=97.3+2.05d−0.0239d²+7.08E−5d³, d∈[20, 150], wherein D is in μm and d is in nm.

Further, in the core-shell structure, the core is tyrosinase loaded with small molecules with anti-inflammatory activity, and the small molecules with anti-inflammatory activity are loaded inside the tyrosinase through hydrophilic and hydrophobic interactions, specifically including, but not limited to, at least one of glaucocalyxin A, vitamin E, lycopene, astaxanthin, hesperidin, and glabridin.

A mass ratio of the small molecules with anti-inflammatory activity to the tyrosinase is 1:1-1000, and the ratio is different according to different small molecules with anti-inflammatory activity. For example, the mass ratio of the small molecules with anti-inflammatory activity to the tyrosinase is 1:2-10, for example, 1:2-5.

Tyrosinase promotes melanin synthesis in melanocytes in the skin and hair follicles, which needs to meet two conditions: the tyrosinase can smoothly penetrate the skin barrier to reach the depth where melanocytes are located; and the tyrosinase can maintain sufficient catalytic activity to catalyze the synthesis of melanin from a substrate. The polymer shell has two functions in this system, namely, promoting transdermal effects and protecting tyrosinase activity, so the thickness regulation of the shell is particularly important. According to our research, the permeability of protein nanogel is related to the particle size, and too thin or too thick a shell is not conducive to the deep delivery of the nanogel. The protective effect of the polymer shell on tyrosinase activity comes from the protection of the protein structure by the shell. Too thin a shell has poor ability to resist severe ambient temperature and pH changes. However, the prerequisite for tyrosinase to play a catalytic role is to contact with the substrate, and an excessively thick shell affects the transport of the substrate, thus hindering the activity of the tyrosinase. In the present disclosure, tyrosinase nanogels with a series of sizes are prepared, the influence of shells with different thicknesses on the transdermal depth and the protection of tyrosinase activity is investigated, and the thickness of a polymer shell suitable for vitiligo treatment is comprehensively selected to be 7-20 nm.

The protein core of the tyrosinase nanogel has a hydrophobic structure. Therefore, for inflammatory vitiligo, small molecules with the anti-inflammatory function can be loaded inside tyrosinase, which plays a synergistic role in the treatment of inflammatory vitiligo from two aspects: reducing the inflammatory response in affected areas and promoting melanin production in situ. In this system, the tyrosinase nanogel, as an efficient transdermal delivery carrier, loads anti-inflammatory small molecules inside the tyrosinase to be delivered as a whole. By optimizing the design of the polymer shell of the tyrosinase nanogel and controlling the transdermal depth of the nanogel, the small molecules and the nanogel are delivered to the location of inflammation together in a targeted manner, thereby achieving a high anti-inflammatory effect with low concentrations of anti-inflammatory small molecules.

A second object of the present disclosure is to provide a preparation method for the tyrosinase-based transdermal delivery system as described above, including the steps of:
(1) mixing a tyrosinase solution with a solution of small molecules with polymerizable carbon-carbon double bonds to obtain tyrosinase modified with polymerizable double bonds on the surface; and
(2) mixing a solution of the tyrosinase modified with polymerizable double bonds on the surface, a monomer and a crosslinking agent, adding an initiator, and initiating a polymerization reaction to obtain the tyrosinase-based transdermal delivery system.

Further, in the step (1), the small molecules with polymerizable carbon-carbon double bonds are selected from at least one of N-propenylsuccinimide and maleimide; and a mass ratio of the tyrosinase to the small molecules with polymerizable carbon-carbon double bonds is 5-20:1, preferably 5-10:1. In the solution of small molecules with polymerizable carbon-carbon double bonds, a solvent is at least one of dimethyl sulfoxide and ethanol, and a mass concentration of the small molecules with polymerizable carbon-carbon double bonds in the solution is 1-5 wt %.

Further, in the step (1), a solvent of the tyrosinase solution is a phosphate buffer solution or an organic solvent; the phosphate buffer solution is NaH₂PO₄—Na₂HPO₄, and the phosphate buffer solution has a pH of 5-8; the organic solvent is at least one selected from ethanol, acetone, and dimethyl sulfoxide; and the concentration of protein in a protein solution is 1-100 mg/L, preferably 1-10 mg/L.

Further, in the step (2), the monomer is at least one selected from acrylamide, 2-methacryloyloxyethylphosphocholine, [2-(methacryloyloxy)ethyl]dimethyl-(3-sulfopropyl) ammonium hydroxide, N-(3-aminopropyl) methacrylamide hydrochloride, vinylpyrrolidone, and polyethylene glycol methyl ether acrylate, preferably N-(3-aminopropyl) methacrylamide hydrochloride; the crosslinking agent is at least one selected from N,N′-methylenebisacrylamide, ethylene glycol diacrylate, polyethylene glycol diacrylate, and glycerol dimethacrylate; the initiator is at least one selected from sodium persulfate, potassium persulfate and ammonium persulfate; the amount of the initiator added is 1-5 mg per milligram of tyrosine in the system; preferably, a co-initiator, such as at least one of sodium bisulfite, potassium bisulfite and tetramethylethylenediamine, is further added, and a mass ratio of the initiator to the co-initiator is 1:1-2.

Preferably, a certain amount of N-(3-aminopropyl) methacrylamide hydrochloride is included in the monomer. The type of the monomer determines the surface electrical properties of the polymer shell. By using N-(3-aminopropyl) methacrylamide hydrochloride as the monomer, the surface of the obtained polymer has a positive charge, which is beneficial to the interaction with lipid components in the skin, weakens the blocking effect of a lipid bilayer on protein nanogel, and promotes the intercellular penetration of the protein nanogel. Preferably, 5-20 mol % of N-(3-aminopropyl) methacrylamide hydrochloride is included in the monomer, preferably 10-15 mol % of N-(3-aminopropyl) methacrylamide hydrochloride is included.

Further, a molar ratio of the tyrosinase modified with polymerizable double bonds on the surface to the monomer to the crosslinking agent is 1:100-100000:10-10000, preferably 1:2000-20000:200-2000.

Further, when the initiator is added for polymerization, the concentration of tyrosinase in a reaction system is 1-5 mg/mL, preferably 1-2 mg/mL.

In a preferred technical solution of the present disclosure, in the step (2), the monomer is a compound of acrylamide and N-(3-aminopropyl) methacrylamide hydrochloride, and the crosslinking agent is N,N′-methylenebisacrylamide; and a feeding molar ratio of the tyrosinase modified with polymerizable double bonds on the surface to acrylamide to N-(3-aminopropyl) methacrylamide hydrochloride to N,N′-methylene bisacrylamide is 1:4500-6600:600-800:450-660.

Further, in the step (2), the polymerization reaction is polymerization at room temperature for 2-10 h, such as 4-6 h.

Preferably, for the system in which tyrosinase is loaded with small molecules with anti-inflammatory activity, a preparation method for the tyrosinase-based transdermal delivery system includes the steps of:
(1) mixing a tyrosinase solution with a solution of small molecules with polymerizable carbon-carbon double bonds to obtain tyrosinase modified with polymerizable double bonds on the surface;
(2) adding a solution of small molecules with anti-inflammatory activity into a solution of the tyrosinase modified with polymerizable double bonds on the surface, and performing uniform mixing to obtain a tyrosinase solution loaded with small molecules with anti-inflammatory activity; and
(3) mixing the tyrosinase solution loaded with small molecules with anti-inflammatory activity, a monomer and a crosslinking agent, adding an initiator, and initiating a polymerization reaction to obtain the tyrosinase-based transdermal delivery system.

That is, the step of loading the small molecules with anti-inflammatory activity with the tyrosinase is added, and the addition of the small molecules with anti-inflammatory activity cooperates with the tyrosinase to further enhance the therapeutic effect on vitiligo.

In the solution of small molecules with anti-inflammatory activity, a solvent is at least one selected from dimethyl sulfoxide, N,N-dimethylformamide, tetrahydrofuran, ethanol and isopropanol, and the concentration of the small molecules with anti-inflammatory activity is 0.1-10 mg/L. A mass ratio of the small molecules with anti-inflammatory activity to the tyrosinase modified with polymerizable double bonds on the surface (in terms of tyrosinase) is 1:1-100. The ratio of the small molecules with anti-inflammatory activity to the tyrosinase mainly depends on the loading amount of the small molecules with anti-inflammatory activity by the tyrosinase, and the ratio varies according to different small molecules with anti-inflammatory activity. For example, the mass ratio of the small molecules with anti-inflammatory activity to the tyrosinase is 1:2-10, for example, 1:2-5.

A third object of the present disclosure is to provide use of the tyrosinase-based transdermal delivery system in the manufacture of a medicament for treating vitiligo. Specifically, transdermal administration is performed on an affected area, such as applying an ointment, a patch, a gel dressing, etc.

The present disclosure achieves the following beneficial effects through the above technical solutions:
1. In the present disclosure, the tyrosinase is modified with compounds with polymerizable double bonds, and a polymerization reaction is initiated in situ on the surface of protein to coat the surface of the protein with a polymer protective layer which is bio-friendly and has no or little irritation to skin. The polymer layer can effectively protect the activity of the tyrosinase and promote the transdermal delivery of the tyrosinase, and by regulating and controlling the polymerization conditions of the polymer shell (including the feeding time, the reaction time, etc.), tyrosinase nanogel can be controlled to be delivered to melanocytes deep in the skin and hair follicles in a targeted manner to promote the in-situ synthesis of melanin.
2. The transdermal delivery system of the present disclosure can also load active small molecules with an anti-inflammatory function at a center of the tyrosinase, and the small molecules are released after being delivered to an inflammatory area of vitiligo in a targeted manner along with the tyrosinase nanogel, so that inflammation is relieved, and the melanocytes are protected from being damaged.
3. The preparation method for the transdermal delivery system of the present disclosure is simple, and the transdermal delivery system has high yield, and can be industrially produced on a large scale.
4. Raw materials used in the present disclosure are all bio-friendly and can be used as reagents for drugs, so they can be used in transdermal delivery systems for ingredients beneficial to the human body such as drugs.

### BRIEF DESCRIPTION OF DRAWING(S)

FIG. 1 is a fitting curve of a penetration depth D versus a particle size d of tyrosinase nanogel with a positive surface potential at 24 hours;

FIG. 2 shows the relative activity of nTYR with different sizes and native tyrosinase;

FIG. 3 is a TEM image of PAAm-nTYR@GLA obtained in Example 2;

FIG. 4 is a TEM image of PMPC-nTYR@GLA obtained in Example 3;

FIG. 5 shows the depigmentation on the back of mice in a progressive stage of vitiligo; and

FIG. 6 shows the staining results of mouse skin sections.

### DETAILED DESCRIPTION

Hereinafter, the present disclosure will be further described with reference to specific examples, but is not limited to the contents of the description. The reagents used are all reagents commercially available in the art.

### Example 1

To 2 mL of a tyrosinase solution with a concentration of 1 mg/mL, 40 mg of a N-propenylsuccinimide solution with a mass concentration of 1 wt % (small molecules with polymerizable carbon-carbon double bonds) was added, with a solvent being dimethyl sulfoxide. Well mixing was performed, and a reaction was carried out for 2 h to obtain tyrosinase modified with polymerizable double bonds on the surface (aTYR).

aTYR, acrylamide (a monomer), N-(3-aminopropyl) methacrylamide hydrochloride (a monomer) and N,N′-methylenebisacrylamide (a crosslinking agent) were mixed at a given molar ratio, the protein concentration was diluted to 1 mg/mL with a phosphate buffer solution having a pH of 7.4, an initiator was then added according to 3.8 mg of ammonium persulfate and 7.6 mg of tetramethylethylenediamine per mg of protein, and in-situ free radical polymerization was initiated at room temperature to obtain polyacrylamide-tyrosinase nanogels (nTYR) with a series of sizes after reacting for a specified duration. The preparation conditions and the measured final sizes and surface zeta potentials are shown in Table 1.

TABLE 1
Preparation conditions, final sizes and surface potentials
of tyrosinase nanogels with a series of sizes

| | Molar mass of | | | Reac- | Parti- | Zeta |
|---|---|---|---|---|---|---|
| Different | monomer/crosslinking agent | | | tion | cle | poten- |
| nanogels | required per mole of aTYR | | | time | size | tial |
| nTYR | AAm | APM | BIS | (h) | (nm) | (mV) |
| nTYR-20− | 5000 | / | 500 | 4 | 20.8 | −6.1 ± 2.3 |
| nTYR-20+ | 4500 | 600 | 450 | 4 | 22.6 | +4.7 ± 0.8 |
| nTYR-50− | 7000 | / | 700 | 4 | 50.5 | −6.0 ± 3.5 |
| nTYR-50+ | 6600 | 800 | 660 | 4 | 47.7 | +5.1 ± 2.2 |
| nTYR-100− | 10000 | / | 1000 | 6 | 97.3 | −5.8 ± 1.7 |
| nTYR-100+ | 10000 | 1000 | 1000 | 6 | 103.2 | +5.6 ± 1.3 |
| nTYR-150− | 13000 | / | 1300 | 8 | 150.9 | −5.5 ± 2.0 |
| nTYR-150+ | 12500 | 1200 | 1250 | 8 | 144.8 | +6.0 ± 2.5 |
| nTYR-200− | 25000 | / | 2500 | 8.5 | 229.2 | −6.9 ± 1.4 |
| nTYR-200 | 22000 | 2500 | 2200 | 8.5 | 241.5 | +4.5 ± 1.8 |

It can be seen from Table 1 that increasing the amount of the monomers used relative to the protein and increasing the polymerization time make the particle sizes of the obtained polyacrylamide-tyrosinase nanogels (nTYR) larger and increase the proportion of APM in the monomers, which makes the Zeta potential of nTYR more positive.

### Application Example 1 Transdermal Experiment of Tyrosinase Nanogels with a Series of Sizes

4 mL of tyrosinase nanogels nTYR with different sizes and having a concentration of 1 mg/mL in Example 1 was mixed with 1 mL of fluorescein isothiocyanate at a concentration of 1 mg/mL, a reaction was carried out overnight, and the resulting reaction solution was dialyzed overnight in a dialysis bag having a molecular weight cutoff of 14,000 Da to remove unreacted small molecules to obtain fluorescently labeled nTYR. Fresh pork skin was washed with PBS to be clean, and was then fixed in a transdermal diffuser with a stratum corneum facing upwards, followed by incubation at 37° C. 0.5 mL of a fluorescently labeled tyrosinase nanogel solution with a concentration of 1 mg/mL was dropwise added to the pig skin tissue, and frozen sections were prepared after 24 hours, and the penetration of the nanogels was observed under a confocal microscope. The statistical penetration depth results are shown in Table 2.

TABLE 2
Transdermal depth of tyrosinase nanogels with a series of sizes at 24 hours

| Sample | | | | | |
|---|---|---|---|---|---|
| name | nTYR-20− | nTYR-50− | nTYR-100− | nTYR-150− | nTYR-200− |
| Transdermal | 79.3 ± 13.1 | 85.4 ± 9.9 | 77.6 ± 11.5 | 69.8 ± 10.2 | 56.1+7.9 |
| depth (μm) | | | | | |
| Sample | | | | | |
| name | nTYR-20+ | nTYR-50+ | nTYR-100+ | nTYR-150+ | nTYR-200+ |
| Transdermal | 128.5 ± 7.8 | 159.5 ± 11.1 | 113.6 ± 9.9 | 99.7 ± 8.5 | 58.4 ± 6.3 |
| depth (μm) | | | | | |

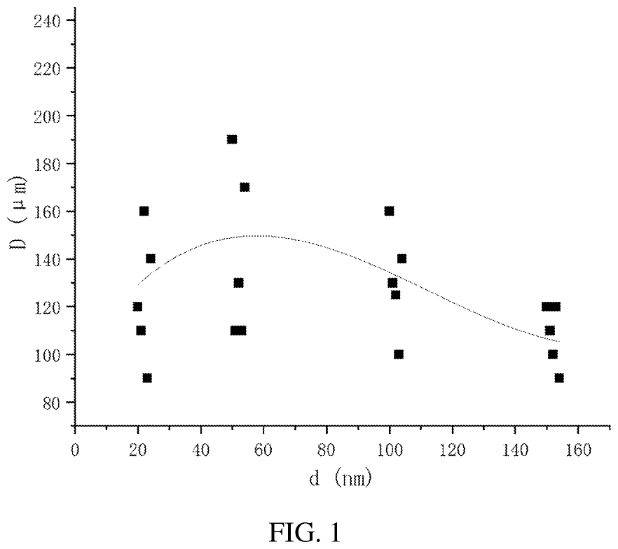

By comparing the penetration depths of tyrosinase nanogels with different sizes and positive surface potentials (about +5 mV), it is found that when the particle size is 20-150 nm, protein nanogels can penetrate the epidermis to reach the vicinity of the dermis within 24 hours, and the penetration depth increases first and then decreases with the increase of the particle size of the gel. However, tyrosinase nanogels larger than 200 nm mostly stay in the stratum corneum in an upper part of the epidermis. The penetration depths of nTYR-20+, nTYR-50+, nTYR-100+, and nTYR-150+ are fitted. FIG. 1 is a fitting curve of a penetration depth D versus a particle size d of tyrosinase nanogel with a positive surface potential at 24 hours. A formula after fitting is as follows:

D=97.3+2.05d−0.0239d²+7.08E−5d³, d∈[20, 150], wherein D is in μm and dis in nm.

This expression can be used to preliminarily predict the penetration ability of tyrosinase nanogels with positive surface potentials and particle sizes ranging from 20 to 150 nm. In the treatment of vitiligo, drug delivery targets are melanocytes located at a junction of the epidermis and the dermis and deep in hair follicles. According to the formula, the final penetration depth of tyrosinase nanogels with sizes of about 20-45 nm and 80-130 nm is near the melanin location, which meets the delivery requirements. Considering the therapeutic efficacy comprehensively, the tyrosinase-based transdermal delivery system of the present disclosure, also referred to as the tyrosinase nanogel, preferably has a size of 20-45 nm.

### Application Example 2 Activity Retention of Tyrosinase Nanogels with a Series of Sizes

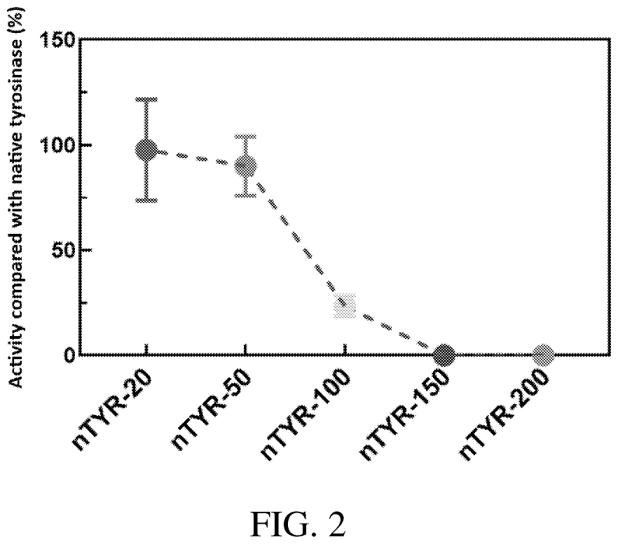

The catalytic activity of nTYR was determined by a UV spectrophotometer. 1600 μL of PBS buffer with a pH of 6 and 1400 μL of a L-tyrosine solution at a concentration of 1 g/L were added to a reference cell; 1550 μL of PBS buffer with a pH of 6 and 1400 μL of a L-tyrosine solution at a concentration of 1 g/L were added to a sample cell, and 50 μL of a nTYR solution or an untreated tyrosinase solution was finally added, the reference cell and the sample cell were placed into the UV spectrophotometer, the change in absorbance of the sample cell at 317 nm over the time in the first 20 minutes was recorded, and a slope of a fitting straight line was used to represent the relative activity of tyrosinase. The results are shown in FIG. 2 and Table 3. It is indicated that an excessively thick polymer shell of tyrosinase nanogel with a size of larger than 50 nm affects the contact between tyrosinase and a substrate, or the polymerization conditions cause the loss of tyrosinase activity. The loss of activity of tyrosinase nanogel with a size of less than 50 nm is within an acceptable range.

TABLE 3
Relative activity of nTYR with different sizes

| Sample | | | | | |
|---|---|---|---|---|---|
| name | nTYR-20 | nTYR-50 | nTYR-100 | nTYR-150 | nTYR-200 |
| Activity | 96.1 ± 8.5 | 89.5 ± 6.9 | 27.3 ± 3.2 | / | / |
| compared | | | | | |
| with native | | | | | |
| tyrosinase | | | | | |
| (%) | | | | | |

### Application Example 3 Thermal Stability Test of Tyrosinase Nanogels with a Series of Sizes

The catalytic activity of nTYR was determined by a UV spectrophotometer. 1600 μL of PBS buffer with a pH of 6 and 1400 μL of a L-tyrosine solution at a concentration of 1 g/L were added to a reference cell; 1550 μL of PBS buffer with a pH of 6 and 1400 μL of a L-tyrosine solution at a concentration of 1 g/L were added to a sample cell, and 50 μL of a nTYR solution or a native tyrosinase solution after different heat treatments was finally added, the reference cell and the sample cell were placed into the UV spectrophotometer, the change in absorbance of the sample cell at 317 nm over the time in the first 20 minutes was recorded, and a slope of a fitting straight line was used to represent the relative activity of tyrosinase. The retention of activity of enzymes after different heat treatments relative to those without heat treatments was calculated, and the results are shown in Table 4.

TABLE 4
Relative activity of a series of nTYR

| | | Native | | |
|---|---|---|---|---|
| | | tyrosinase | | |
| Sample name | | (TYR) | nTYR-20 | nTYR-50 |
| Activity | Treatment at | 90.3 ± 11.9 | 92.1 ± 3.4 | 93.0 ± 5.2 |
| retention after | 40 °C for 0.5 h | | | |
| heat treatment | Treatment at | 75.3 ± 6.0 | 87.8 ± 1.2 | 76.9 ± 2.9 |
| (%) | 40 °C for 1 h | | | |
| | Treatment at | 13.6 ± 0.9 | 46.8 ± 1.8 | 43.2 ± 5.8 |
| | 60 °C for 0.5 h | | | |
| | Treatment at | 0.6 ± 0.58 | 41.2 ± 6.7 | 37.5 ± 5.2 |
| | 60 °C for 1 h | | | |

It was found that the native tyrosinase was significantly inactivated at 60° C., while nTYR-20 and nTYR-50 could still retain 40% activity after treatment at 60° C. for 1 h, indicating the better thermal protection effect of the polymer shell.

### Application Example 4: Acid-Base Stability Test of Tyrosinase Nanogels with a Series of Sizes

The catalytic activity of nTYR was determined by a UV spectrophotometer. 1600 μL of PBS buffer with a specified pH and 1400 μL of a L-tyrosine solution at a concentration of 1 g/L were added to a reference cell; 1550 μL of PBS buffer with a specified pH and 1400 μL of a L-tyrosine solution at a concentration of 1 g/L were added to a sample cell, and 50 μL of a nTYR solution or a native tyrosinase solution after different heat treatments was finally added, the reference cell and the sample cell were placed into the UV spectrophotometer, the change in absorbance of the sample cell at 317 nm over the time in the first 20 minutes was recorded, and a slope of a fitting straight line was used to represent the relative activity of tyrosinase. The retention of activity of enzymes at different pHs relative to that at pH-6 was calculated. The results are shown in Table 5.

TABLE 5
Relative activity of a series of nTYR

| | | Native | | |
|---|---|---|---|---|
| | | tyrosinase | | |
| Sample name | | (TYR) | nTYR-20 | nTYR-50 |
| Activity | pH = 4 | 14.4 ± 2.5 | 52.6 ± 7.8 | 40.4 ± 1.9 |
| retention (%) | pH = 5 | 57.7 ± 5.0 | 78.7 ± 1.9 | 70.5 ± 14.9 |
| | pH = 6 | 100.0 ± 9.9 | 100.0 ± 9.4 | 100.0 ± 20.2 |
| | pH = 7 | 66.1 ± 8.8 | 100.1 ± 20.1 | 113.1 ± 6.0 |
| | pH = 8 | 40.3 ± 9.4 | 92.1 ± 12.1 | 100.1 ± 6.4 |

It was found that the native tyrosinase had good activity only in a weakly acidic environment (with a pH of around 6), while nTYR-20 and nTYR-50 could exert efficient catalytic effects in a wide pH range, indicating that the polymer shell provided better acid and alkali resistance for nTYR.

### Example 2 Preparation of Tyrosinase Nanogel Loaded with Glaucocalyxin A

(1) To 2 mL of a tyrosinase solution with a concentration of 1 mg/mL, 40 mg of a N-propenylsuccinimide solution with a mass concentration of 1 wt % was added, with a solvent being dimethyl sulfoxide. Well mixing was performed, and a reaction was carried out for 2 hours to obtain tyrosinase modified with polymerizable double bonds on the surface (aTYR).
(2) 2 mL of the 1 mg/mL tyrosinase solution modified with double bonds on the surface was taken, 0.5 mL of a 2 mg/mL DMSO solution of glaucocalyxin A was added dropwise to the tyrosinase solution under the condition of 300 r/min while stirring, and thorough mixing was performed to obtain tyrosinase loaded with glaucocalyxin A (aTYR@GLA).
(3) aTYR@GLA, acrylamide, N-(3-aminopropyl) methacrylamide hydrochloride and N,N′-methylenebisacrylamide were mixed at a molar ratio of 1:6000:600:600, the protein concentration was diluted to 1 mg/mL with a phosphate buffer solution having a pH of 7.4, an initiator was then added according to 3.8 mg of ammonium persulfate and 7.6 mg of tetramethylethylenediamine per mg of protein, and in-situ free radical polymerization was initiated at room temperature to obtain polyacrylamide-tyrosinase nanogel loaded with glaucocalyxin A (PAAm-nTYR@GLA) after reacting for 4 h.

The obtained compound solution was subjected to ultrafiltration treatment in an ultrafiltration centrifuge tube having a molecular weight cutoff of 30 kDa to obtain a purified high-concentration mother liquor having a concentration of 6.1 mg/mL, and the mother liquor was stored under refrigeration.

### Example 3 Preparation of Polyphosphocholine-Tyrosinase Nanogel Loaded with Glaucocalyxin A

(1) To 2 mL of a tyrosinase solution with a concentration of 1 mg/mL, 40 mg of a N-propenylsuccinimide solution with a mass concentration of 1 wt % was added, with a solvent being dimethyl sulfoxide. Well mixing was performed, and a reaction was carried out for 2 hours to obtain tyrosinase modified with polymerizable double bonds on the surface (aTYR).
(2) 2 mL of the 1 mg/mL tyrosinase solution modified with double bonds on the surface was taken, 0.5 mL of a 2 mg/mL DMSO solution of glaucocalyxin A was added dropwise to the tyrosinase solution under the condition of 300 r/min while stirring, and thorough mixing was performed to obtain tyrosinase loaded with glaucocalyxin A (aTYR@GLA).
(3) aTYR@GLA, 2-methacryloyloxyethylphosphocholine, and N,N′-methylenebisacrylamide were mixed at a molar ratio of 1:40:4, the protein concentration was diluted to 1 mg/mL with a phosphate buffer solution having a pH of 7.4, an initiator was then added according to 3.8 mg of ammonium persulfate and 7.6 mg of tetramethylethylenediamine per mg of protein, and in-situ free radical polymerization was initiated at room temperature to obtain polyphosphocholine-tyrosinase nanogel loaded with glaucocalyxin A (PMPC-nTYR@GLA) after reacting for 4 h.

The obtained compound solution was subjected to ultrafiltration treatment in an ultrafiltration centrifuge tube having a molecular weight cutoff of 30 kDa to obtain a purified high-concentration mother liquor having a concentration of 5.7 mg/mL, and the mother liquor was stored under refrigeration.

Characterization of the size of the nanogel by dynamic light scattering (DLS): the protein nanogel solutions at a concentration of 1 mg/mL in Examples 2 and 3 were added to a sample cell, and the particle size of a compound in the system was tested at room temperature by using a Malvern Nano Zs90 nanoparticle size potentiometer. The results are statistically shown in Table 6.

TABLE 6
Particle size and Zeta potential of tyrosinase nanogel

| | Particle size distribution | |
|---|---|---|
| Sample name | center (nm) | Zeta potential (mV) |
| TYR | 6 | −6.4 ± 2.1 |
| PAAm-nTYR@GLA | 33 | +4.8 ± 1.5 |
| PMPC-nTYR@GLA | 41 | −4.3 ± 3.0 |

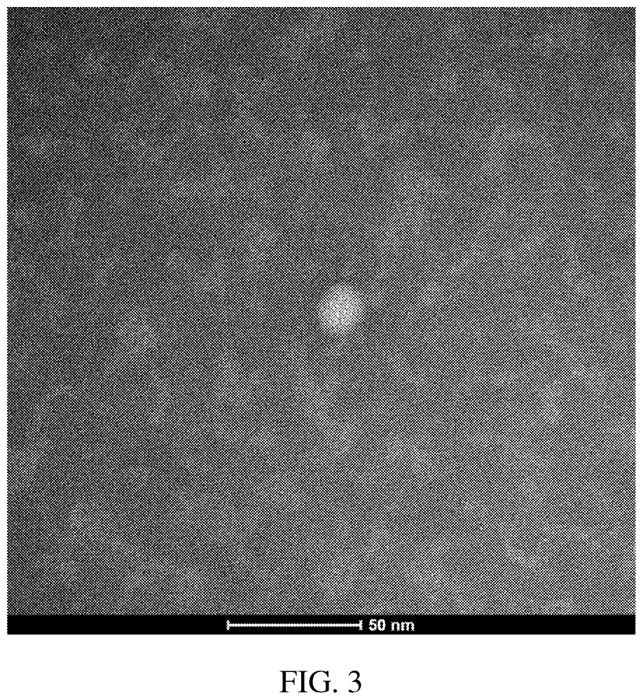

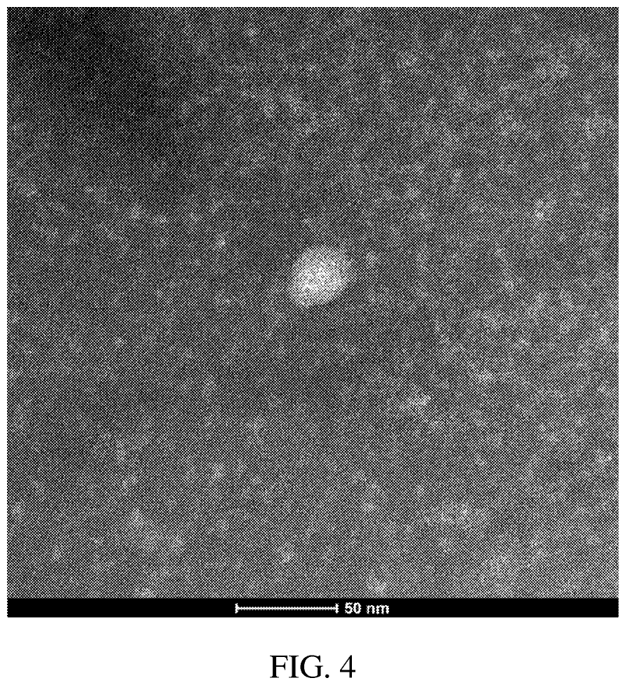

Characterization of the morphology of the nanogel by transmission electron microscopy (TEM): the morphology and dispersion of PAAm-nTYR@GLA under a transmission electron microscope are shown in FIG. 3. The morphology and dispersion of PMPC-nTYR@GLA under a transmission electron microscope are shown in FIG. 4. It can be seen that the particle size of the tyrosinase nanogel is consistent with DLS results, and the tyrosinase nanogel is close to a spherical shape as a whole, with good dispersion.

### Application Example 5

2 mL of the tyrosinase nanogel at a concentration of 5 mg/mL prepared in Examples 2 and 3 was placed into a dialysis bag having a molecular weight cutoff of 3000, and the dialysis bag was immersed in a phosphate buffer solution with a pH of 6, and the entire system was shaken at 37° C. The sustained release time was 48 h. Within 48 h, samples were taken at regular intervals, and the concentration of loaded small molecule drugs was measured under an ultraviolet spectrophotometer, and the sustained release of small molecule drugs by a composite system was calculated. The results are statistically shown in Table 7.

TABLE 7
Release of small molecule drugs from tyrosinase nanogels

Cumulative release rate (%)

| Time (h) | PAAm-nTYR@GLA | PMPC-nTYR@GLA |
|---|---|---|
| 0.25 | 9.3 ± 1.1 | 5.9 ± 2.1 |
| 0.5 | 20.9 ± 5.6 | 18.3 ± 5.7 |
| 1 | 35.8 ± 3.7 | 33.1 ± 2.6 |
| 2 | 46.7 ± 9.2 | 45.9 ± 4.8 |
| 4 | 66.5 ± 2.9 | 69.8 ± 6.5 |
| 8 | 78.8 ± 6.3 | 76.9 ± 4.3 |
| 24 | 80.9 ± 2.3 | 78.9 ± 1.8 |

### Application Example 6 Validation of the Efficacy of Functional Small Molecule Components

Determination of the anti-inflammatory ability of the polyacrylamide-tyrosinase nanogel (PAAm-nTYR@GLA) in Example 2 and the polyphosphocholine-tyrosinase nanogel (PMPC-nTYR@GLA) in Example 3: the NO concentration in inflammatory cells was detected by using a Griess kit. HaCaT cells (1*10⁵ cells/well) were inoculated in a 24-well plate. After 12 h, when the cells were completely adhered to the wall, the original medium was sucked away. 100 μL of a serum-free medium was added to all groups, 10 μL of a serum-free medium was added to a negative control group, 10 μL of PBS buffer was added to a positive control group, 10 μL of a nanogel solution with a concentration of 0.01 mg/mL was added to a low-concentration experimental group, 10 μL of a nanogel solution with a concentration of 0.02 mg/mL was added to a medium-concentration experimental group, and 10 μL of a nanogel solution with a concentration of 0.04 mg/mL was added to a high-concentration experimental group. After co-incubation for 2 h, the original medium was sucked away from all groups, 110 μL of a fresh medium was added to the negative control group, and 100 μL of a fresh medium and 10 μL of interleukin-1ß at a concentration of 10 ng/ml were added to the remaining groups. After co-incubation for 24 h, the determination was carried out according to the instructions of the Griess kit. The results are statistically shown in Table 8. It was found that both PMPC-nTYR@GLA and PAAm-nTYR@GLA had good anti-inflammatory ability, and this ability increased with the increase of concentration. The positive charge on the surface of PAAm-nTYR@GLA makes it more efficiently endocytosed by the cells, and PAAm-nTYR@GLA has better anti-inflammatory ability than the negatively charged PMPC-nTYR@GLA at the same concentration.

TABLE 8
Improvement of the intracellular anti-inflammatory ability
of tyrosinase nanogel with the increase of concentration

| Experimental group | | NO concentration (μM) |
|---|---|---|
| Negative control group | | 21.6 ± 5.8 |
| Positive control group (PBS) | | 69.8 ± 5.9 |
| PMPC-nTYR@GLA | Low concentration | 61.2 ± 1.7 |
| | Medium concentration | 52.3 ± 4.8 |
| | High concentration | 39.5 ± 7.0 |
| PAAm-nTYR@GLA | Low concentration | 58.5 ± 4.3 |
| | Medium concentration | 46.1 ± 3.2 |
| | High concentration | 30.4 ± 6.5 |

### Application Example 7 Cell Experiment for Promoting Melanogenesis

Determination of the effect of the polyphosphocholine-tyrosinase nanogel (PMPC-nTYR@GLA) in Example 3 on melanin synthesis in mouse melanoma B16F10 cells: B16F10 cells (1*10⁵ cells/well) were inoculated in a 24-well plate. After 12 h, when the cells were completely adhered to the wall, the original medium was sucked away from all groups, and 100 μL of a serum-free medium was added. 10 μL of PBS buffer was added to a control group, 10 μL of a nanogel solution with a concentration of 0.01 mg/mL was added to a low-concentration experimental group, 10 μL of a nanogel solution with a concentration of 0.02 mg/mL was added to a medium-concentration experimental group, and 10 μL of a nanogel solution with a concentration of 0.04 mg/mL was added to a high-concentration experimental group. After co-incubation for 48 h, the cells in all groups were separately collected and dissolved in 1 mL of a NaOH solution (1 mol/L, containing 10% DMSO), and the cells were ultrasonic disrupted for 30 minutes, placed in a water bath at 80° C. for 30 minutes, and centrifuged at 3000 r/min for 15 minutes. A supernatant was taken, and an absorbance at 450 nm was measured. The results are shown in Table 9. It was found that PMPC-nTYR@GLA had a good melanogenesis-promoting ability, and this ability increased with the increase of concentration.

TABLE 9
Improvement of the melanogenesis-promoting ability of
tyrosinase nanogel with the increase of concentration

| | | Relative melanin content |
|---|---|---|
| Experimental group | | (%) |
| Control group (PBS) | | 100.0 ± 5.9 |
| PMPC-nTYR@GLA | Low concentration | 105.2 ± 4.7 |
| | Medium concentration | 117.3 ± 8.1 |
| | High concentration | 140.2 ± 7.9 |
| PAAm-nTYR@GLA | Low concentration | 108.6 ± 5.3 |
| | Medium concentration | 125.4 ± 2.9 |
| | High concentration | 148.7 ± 3.1 |

### Application Example 8 Transdermal Animal Experiment on Vitiligo Model

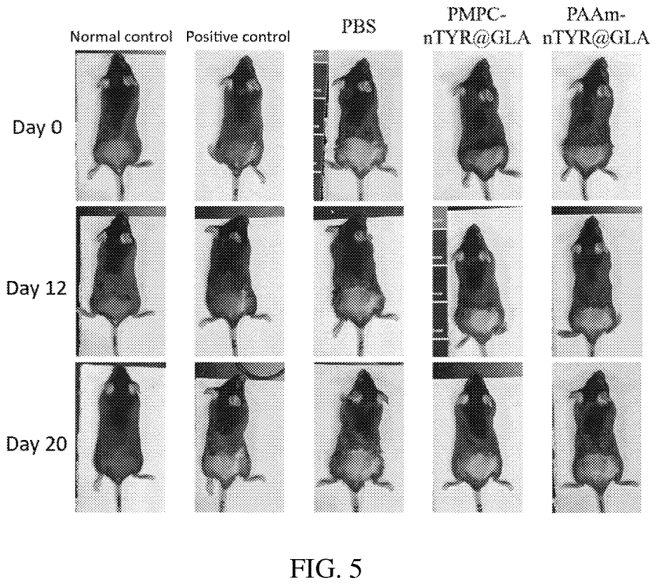

The ability of transdermal delivery of PAAm-nTYR@GLA and PMPC-nTYR@GLA in Examples 2 and 3 to promote pigment production was investigated by using a vitiligo mouse model. The mice were randomly divided into 5 groups, and the backs were subjected to unified hair removal treatment, one group was used as a normal control, and the remaining 4 groups (a positive control group, a PBS control group, a PAAm-nTYR@GLA group and a PMPC-nTYR@GLA group) were subjected to vitiligo modeling. After successful modeling, the corresponding drug or PBS buffer (the positive control group) was applied to the hair removal areas every two days, and hair on the back was collected before each administration and photographed to record the overall pigmentation. After 20 consecutive days of administration, skin on the back of the mice was taken, and the overall pigmentation on the back of the mice was observed to characterize the incidence of vitiligo. The results are shown in FIG. 5. It was found that the incidence of vitiligo on the back deteriorated significantly after 20 days in the untreated positive control group, while the deterioration of vitiligo symptoms on the back in the groups treated with the tyrosinase nanogels was weaker than that in the PBS group, and the treatment effect of the group of PAAm-nTYR@GLA with a positive surface charge was better than that of the group of PMPC-nTYR@GLA with a negative surface potential.

TABLE 10
Statistics on the increase in overall depigmentation
color difference and depigmentation area

| | Increase in | Growth rate of |
|---|---|---|
| | depigmentation color | depigmentation |
| Experimental group | difference value | area ratio |
| Positive control group | 22.6 ± 5.3 | 89.4 ± 6.2 |
| PBS | 17.8 ± 8.3 | 88.1 ± 8.2 |
| PMPC-nTYR@GLA | 9.4 ± 3.8 | 61.5 ± 4.1 |
| PAAm-nTYR@GLA | 5.9 ± 1.7 | 35.8 ± 9.5 |

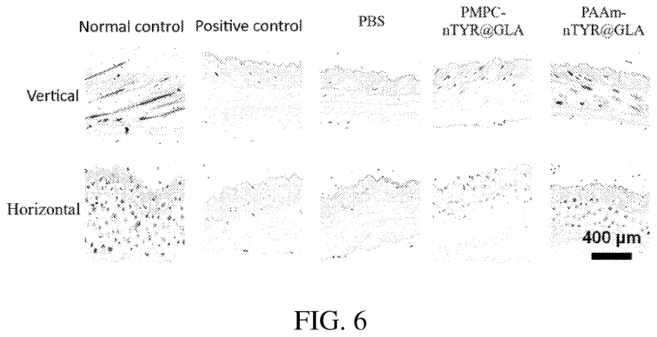

Skin tissue sections were taken to observe hair follicle development and pigmentation, and the results are shown in FIG. 6 and Table 11. It was found that in the positive control group and the PBS group, the number of hair follicles was less, the development was poor, and no pigmented hair shafts were found in the hair follicles. In the groups treated with the tyrosinase nanogels, the development of the hair follicles was better than that in the PBS group. By comparing the PAAm-nTYR@GLA group with the PMPC-nTYR@GLA group, it was found that the pigmentation of hair shafts in the hair follicles in the PAAm-nTYR@GLA group was more obvious. It is indicated that the better transdermal delivery ability brought by the positive charge on the surface of PAAm-nTYR@GLA can deliver tyrosinase to melanocytes in a targeted manner, thereby promoting melanin production, and having a good improvement effect on vitiligo symptoms.

TABLE 11
Statistics on the length and number of hair follicles

| | Average length | Average number |
|---|---|---|
| | of hair follicles | of hair follicles |
| Experimental group | (mm) | (pcs/mm²) |
| Normal control group | 0.335 ± 0.078 | 59.8 ± 6.3 |
| Positive control group | 0.161 ± 0.041 | 28.8 ± 5.9 |
| PBS | 0.156 ± 0.037 | 29.1 ± 4.8 |
| PMPC-nTYR@GLA | 0.223 ± 0.066 | 44.1 ± 8.5 |
| PAAm-nTYR@GLA | 0.290 ± 0.072 | 36.9 ± 7.0 |

1. A tyrosinase-based transdermal delivery system for treating vitiligo, wherein the system is of a core-shell structure, a core is tyrosinase or tyrosinase that adsorbs small molecules with anti-inflammatory activity, and a shell is a polymer shell.

2. The tyrosinase-based transdermal delivery system for treating vitiligo according to claim 1, wherein a polymer in the polymer shell is a polymeric material wrapped around an outer layer of the tyrosinase by a polymerization reaction and comprising at least one of an acrylamide copolymer, a vinylpyrrolidone copolymer, polyol, polylactic acid, a phosphocholine polymer, a betaine polymer, and chitosan, the polymerization reaction being polymerization of monomers with carbon-carbon unsaturated double bonds, or polycondensation or ring-opening polymerization of monomers with different functional groups;
or a polymeric material wrapped around an outer layer of the tyrosinase by electrostatic interaction and being a positively or negatively charged polyelectrolyte, wherein the positively charged polyelectrolyte is at least one selected from poly(acrylamine hydrochloride), poly (L-lysine), polyethyleneimine, poly (L-histidine), poly (N,N-dimethylaminoethyl methacrylate), polymethacrylamidopropyltrimethylammonium chloride, and natural or synthetic polysaccharides; and the negatively charged polyelectrolyte is at least one selected from polyacrylic acid, polystyrene sulfonate, alginate, hyaluronic acid, heparin, heparan sulfate, chondroitin sulfate, dextran sulfate, polymethacrylic acid, oxidized cellulose, carboxymethyl cellulose, polyaspartic acid, and polyglutamic acid.

3. The tyrosinase-based transdermal delivery system for treating vitiligo according to claim 2, wherein a monomer of the polymeric material wrapped around the outer layer of the tyrosinase by the polymerization reaction is at least one selected from vinylpyrrolidone, acrylamide, 2-methacryloyloxyethylphosphocholine, ethylene glycol, propylene glycol, butylene glycol, and lactide.

4. The tyrosinase-based transdermal delivery system for treating vitiligo according to claim 1, wherein in the core-shell structure, the tyrosinase loaded with small molecules with anti-inflammatory activity as the core has a size of 5-15 nm; and the polymer shell as the shell has a thickness of 1-100 nm.

5. The tyrosinase-based transdermal delivery system for treating vitiligo according to claim 1, wherein in the core-shell structure, the tyrosinase loaded with small molecules with anti-inflammatory activity has a size of 5-10 nm; and the polymer shell as the shell has a thickness of 5-50 nm.

6. The tyrosinase-based transdermal delivery system for treating vitiligo according to claim 1, wherein the tyrosinase-based transdermal delivery system has a surface Zeta potential of +4 mV to +6 mV.

7. The tyrosinase-based transdermal delivery system for treating vitiligo according to claim 1, wherein in the core-shell structure, the small molecules with anti-inflammatory activity are loaded inside the tyrosinase by hydrophilic and hydrophobic interactions, and comprise at least one of glaucocalyxin A, vitamin E, lycopene, astaxanthin, hesperidin, and glabridin; and
a mass ratio of the small molecules with anti-inflammatory activity to the tyrosinase is 1:1-1000.

8. A method for regulating and controlling the transdermal depth of the tyrosinase-based transdermal delivery system according to claim 6, wherein the surface Zeta potential is +4 mV to +6 mV, a penetration depth is regulated and controlled by regulating a dimension d of the transdermal delivery system, and the penetration depth D and the dimension d of the transdermal delivery system satisfy the following formula:
D=97.3+2.05d−0.0239d²+7.08E−5d³, d∈[20, 150], wherein D is in μm and d is in nm; and
the method is for the non-therapeutic/diagnostic purpose.

9. A preparation method for the tyrosinase-based transdermal delivery system according to claim 1, comprising the steps of:
(1) mixing a tyrosinase solution with a solution of small molecules with polymerizable carbon-carbon double bonds to obtain tyrosinase modified with polymerizable double bonds on the surface; and
(2) mixing a solution of the tyrosinase modified with polymerizable double bonds on the surface, a monomer and a crosslinking agent, adding an initiator, and initiating a polymerization reaction to obtain the tyrosinase-based transdermal delivery system.

10. The preparation method according to claim 9, wherein in the step (1), the small molecules with polymerizable carbon-carbon double bonds are selected from at least one of N-propenylsuccinimide and maleimide; and a mass ratio of the tyrosinase to the small molecules with polymerizable carbon-carbon double bonds is 5-20:1.

11. The preparation method according to claim 10, wherein in the step (1), the mass ratio of the tyrosinase to the small molecules with polymerizable carbon-carbon double bonds is 5-10:1, and a solvent of the tyrosinase solution is a phosphate buffer solution or an organic solvent; a solute of the phosphate buffer solution is NaH₂PO₄—Na₂HPO₄, and the phosphate buffer solution has a pH of 5-8; the organic solvent is at least one selected from ethanol, acetone, and dimethyl sulfoxide; and the concentration of protein in a protein solution is 1-100 mg/L.

12. The preparation method according to claim 9, wherein in the step (2), the monomer is at least one selected from acrylamide, 2-methacryloyloxyethylphosphocholine, [2-(methacryloyloxy)ethyl]dimethyl-(3-sulfopropyl) ammonium hydroxide, N-(3-aminopropyl) methacrylamide hydrochloride, vinylpyrrolidone, and polyethylene glycol methyl ether acrylate; the crosslinking agent is at least one selected from N,N′-methylenebisacrylamide, ethylene glycol diacrylate, polyethylene glycol diacrylate, and glycerol dimethacrylate; the initiator is at least one selected from sodium persulfate, potassium persulfate and ammonium persulfate; and the amount of the initiator added is 1-5 mg per milligram of tyrosine in the system.

13. The preparation method according to claim 12, wherein the small molecules with polymerizable carbon-carbon double bonds are selected from at least one of N-propenylsuccinimide and maleimide; a mass ratio of the tyrosinase to the small molecules with polymerizable carbon-carbon double bonds is 5-20:1; and
in the step (2), the monomer is N-(3-aminopropyl) methacrylamide hydrochloride; a co-initiator of at least one of sodium bisulfite, potassium bisulfite and tetramethylethylenediamine is also added, and a mass ratio of the initiator to the co-initiator is 1:1-2.

14. The preparation method according to claim 9, wherein in the step (2), the monomer comprises 5-20 mol % of N-(3-aminopropyl) methacrylamide hydrochloride.

15. The preparation method according to claim 9, wherein in the step (2), a molar ratio of the tyrosinase modified with polymerizable double bonds on the surface to the monomer to the crosslinking agent is 1:2000-20000:200-2000.

16. The preparation method according to claim 9, wherein in the step (2), the monomer is a compound of acrylamide and N-(3-aminopropyl) methacrylamide hydrochloride, and the crosslinking agent is N,N′-methylenebisacrylamide; and a feeding molar ratio of the tyrosinase modified with polymerizable double bonds on the surface to acrylamide to N-(3-aminopropyl) methacrylamide hydrochloride to N,N′-methylenebisacrylamide is 1:4500-6600:600-800:450-660.

17. The preparation method according to claim 9, wherein in the step (2), the polymerization reaction is polymerization at room temperature for 2-10 h.

18. A preparation method for the tyrosinase-based transdermal delivery system according to claim 9, comprising the steps of:
(1) mixing a tyrosinase solution with a solution of small molecules with polymerizable carbon-carbon double bonds to obtain tyrosinase modified with polymerizable double bonds on the surface;
(2) adding a solution of small molecules with anti-inflammatory activity into a solution of the tyrosinase modified with polymerizable double bonds on the surface, and performing uniform mixing to obtain a tyrosinase solution loaded with small molecules with anti-inflammatory activity; and
(3) mixing the tyrosinase solution loaded with small molecules with anti-inflammatory activity, a monomer and a crosslinking agent, adding an initiator, and initiating a polymerization reaction to obtain the tyrosinase-based transdermal delivery system.

19. The preparation method according to claim 18, wherein in the solution of small molecules with anti-inflammatory activity, a solvent is at least one selected from dimethyl sulfoxide, N,N-dimethylformamide, tetrahydrofuran, ethanol and isopropanol, and the concentration of the small molecules with anti-inflammatory activity is 0.1-10 mg/mL; and a mass ratio of the small molecules with anti-inflammatory activity to the tyrosinase modified with polymerizable double bonds on the surface (in terms of tyrosinase) is 1:1-100.

20. Use of the tyrosinase-based transdermal delivery system accord to claim 1 in the manufacture of a medicament for treating vitiligo.
